(19) United States
(12) Patent Application Publication
(10) Pub. No.: US 2017/0020671 A1
(43) Pub. Date: Jan. 26, 2017

(54) ACTUATED EXTRA-VENOUS VALVE

(71) Applicant: BOSTON SCIENTIFIC SCIMED INC., Maple Grove, MN (US)

(72) Inventors: Raed N. Rizq, Maple Grove, MN (US); Derek C. Sutermeister, Ham Lake, MN (US)

(73) Assignee: BOSTON SCIENTIFIC SCIMED INC., Maple Grove, MN (US)

(21) Appl. No.: 15/213,954

(22) Filed: Jul. 19, 2016

Related U.S. Application Data
(60) Provisional application No. 62/195,675, filed on Jul. 22, 2015.

(51) Int. Cl. A61F 2/24; A61M 1/10

(52) U.S. Cl. CPC A61F 2/2475; A61M 1/1087; A61F 2250/0001; A61F 2250/001; A61F 2230/0006; A61F 2210/0014; A61M 2230/04; A61M 2230/205; A61M 2230/42

(57) ABSTRACT

An extravenous valve may include a constricting member configured to surround a body lumen or blood vessel and actuate between a lumen or vessel-occluding configuration and a non-lumen or non-vessel-occluding, configuration, the constricting member including a base material and a shape memory material coupled to the base material, a power source in communication with the constricting member, and a controller configured to receive bio-feedback from a patient. The controller may reversibly actuate the constricting member between the lumen or vessel-occluding configuration and the non-lumen or non-vessel-occluding configuration in response to the bio-feedback. The constricting member may be sized and configured to be delivered to the treatment location through the body lumen or blood vessel.

### CROSS-REFERENCE TO RELATED APPLICATIONS

This application claims the benefit of U.S. Provisional Application No. 62/195,675, filed Jul. 22, 2015.

### TECHNICAL FIELD

The disclosure is directed to a medical device for aiding in the treatment of chronic venous disease. More particularly, the disclosure is directed to a medical device configured to assist or replace a malfunctioning venous valve.

### BACKGROUND

Chronic venous disease (otherwise termed chronic venous insufficiency or CVI) is a common and progressive disorder that affects the venous system of the lower extremities with venous hypertension causing various pathologies including pain, swelling, edema, skin changes, and ulcerations. The peripheral venous system functions as a reservoir to store blood and as a conduit to return blood to the heart. Proper functioning of the peripheral venous system depends on a series of valves and muscle pumps. Failure of the venous valves causes blood to flow backwards (reflux) in the veins and pool in the legs. Obstruction of a vein from clots or malformations can likewise result in slow drainage of the veins and increased venous pressure. Some previous attempts at venous valve repair have been made using intra-venous treatments or solutions, which treatments may have and/or create their own risks and/or challenges. Accordingly, there is an ongoing need for alternative medical devices and methods for treating chronic venous disease.

### SUMMARY

The disclosure is directed to several alternative designs, materials and methods of manufacturing medical device structures and assemblies, and uses thereof.

In a first aspect, an extravenous valve may comprise a constricting member configured to surround a blood vessel and actuate between a vessel-occluding configuration and a non-vessel-occluding configuration, the constricting member including a base material and a shape memory material coupled to the base material, a power source in communication with the constricting member, and a controller configured to receive bio-feedback from a patient. The controller reversibly actuates the constricting member between the vessel-occluding configuration and the non-vessel-occluding configuration in response to the bio-feedback.

In addition or alternatively, and in a second aspect, the controller continuously actuates the constricting member back and forth between the vessel-occluding configuration and the non-vessel-occluding configuration.

In addition or alternatively, and in a third aspect, the base material is a polymer. In addition or alternatively, and in a fourth aspect, the shape memory material is at least partially embedded within the base material.

In addition or alternatively, and in a fifth aspect, the power source communicates with the constricting member via one or more wires.

In addition or alternatively, and in a sixth aspect, the power source communicates with the constricting member wirelessly.

In addition or alternatively, and in a seventh aspect, the constricting member is fixedly attachable to the blood vessel at a first end.

In addition or alternatively, and in an eighth aspect, the first end includes an anchoring means.

In addition or alternatively, and in a ninth aspect, the shape memory material is self-biased toward a configuration capable of encircling the blood vessel.

In addition or alternatively, and in a tenth aspect, the power source is carried by the patient.

In addition or alternatively, and in an eleventh aspect, the constricting member is configured to pump blood through the blood vessel.

In addition or alternatively, and in a twelfth aspect, a percutaneously-implantable extravenous valve may comprise a constricting member configured to surround an outer wall of a body lumen of a patient at a treatment location and actuate between a lumen occluding configuration and a non-lumen-occluding configuration, the constricting member including a base material and a shape memory material coupled to the base material, a power source in communication with the constricting member, and a controller configured to continuously actuate the constricting member between the lumen-occluding configuration and the non-lumen-occluding configuration. The constricting member is sized and configured to be delivered to the treatment location through the body lumen.

In addition or alternatively, and in a thirteenth aspect, the controller is configured to receive bio-feedback from the patient.

In addition or alternatively, and in a fourteenth aspect, the controller is configured to continuously actuate the constricting member between the lumen-occluding configuration and the non-lumen-occluding configuration in response to the bio-feedback.

In addition or alternatively, and in a fifteenth aspect, the bio-feedback includes one or more of blood pressure, heart rate, respiration rate, and oxygenation,

In addition or alternatively, and in a sixteenth aspect, a method of treating a defective venous valve may comprise introducing a delivery sheath into a lumen of a blood vessel having a defective venous valve, the delivery sheath having a constricting member of an extravenous valve disposed within a distal end thereof, advancing the distal end of the delivery sheath to a position adjacent the defective venous valve, puncturing a wall of the blood vessel adjacent the defective venous valve, positioning the distal end of the delivery sheath adjacent the puncture, delivering the constricting member out the distal end of the delivery sheath through the puncture, wherein the constricting member is configured to surround the blood vessel adjacent an exterior surface of the blood vessel, and continuously actuating the constricting member between a vessel-occluding configuration and a non-vessel-occluding configuration.

In addition or alternatively, and in a seventeenth aspect, the constricting member includes a base material and a shape memory material coupled to the base material.

In addition or alternatively, and in an eighteenth aspect, the extravenous valve includes a power source and a controller in communication with the constricting member.

In addition or alternatively, and in a nineteenth aspect, a method may include receiving bio-feedback with the controller, and continuously actuating the constricting member between the vessel-occluding configuration and the non-vessel-occluding configuration in response to the bio-feedback.

In addition or alternatively, and in a twentieth aspect, a method may include anchoring a first end of the constricting member to the blood vessel.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The aspects of the disclosure may be more completely understood in consideration of the following detailed description of various embodiments in connection with the accompanying drawings, in which:

FIG. 1 is a partial section view of a portion of a body lumen and an example medical device disposed therein;

FIG. 2 is a partial section view of a percutaneous delivery of an example medical device;

FIG. 3 illustrates an example medical device in a non-vessel-occluding configuration;

FIG. 4 illustrates an example medical device in a non-vessel-occluding configuration;

FIG. 5 illustrates an example medical device in a vessel-occluding configuration;

FIG. 6 illustrates an example medical device in a vessel-occluding configuration; and

FIGS. 7-8 illustrate aspects of an example medical device.

While the aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g. 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Relative terms such as “proximal”, “distal”, “advance”, “retract”, variants thereof, and the like, may be generally be considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. Other relative terms, such as “upstream” and “downstream” refer to a direction of fluid flow within a lumen, such as a body lumen or blood vessel.

The term “vessel-occluding” may be understood to be an inclusive term encompassing a total occlusion of the vessel and/or a partial occlusion of the vessel. In other words, the term “vessel-occluding” may be used interchangeably herein with the term(s) “completely vessel-occluding”, “totally vessel-occluding”, and/or “partially vessel-occluding”. “Partially vessel-occluding” may encompass any and/or all degrees of occlusion less than a complete or total occlusion. In some instances where the term “vessel-occluding” is used, for example with respect to a venous valve, some leakage of fluid and/or blood through the valve may be permitted in a “vessel-occluding” configuration or arrangement. In some instances where the term “vessel-occluding” is used, for example with respect to a venous valve, no leakage of fluid and/or blood through the valve may be permitted in a “vessel-occluding” configuration or arrangement.

it is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

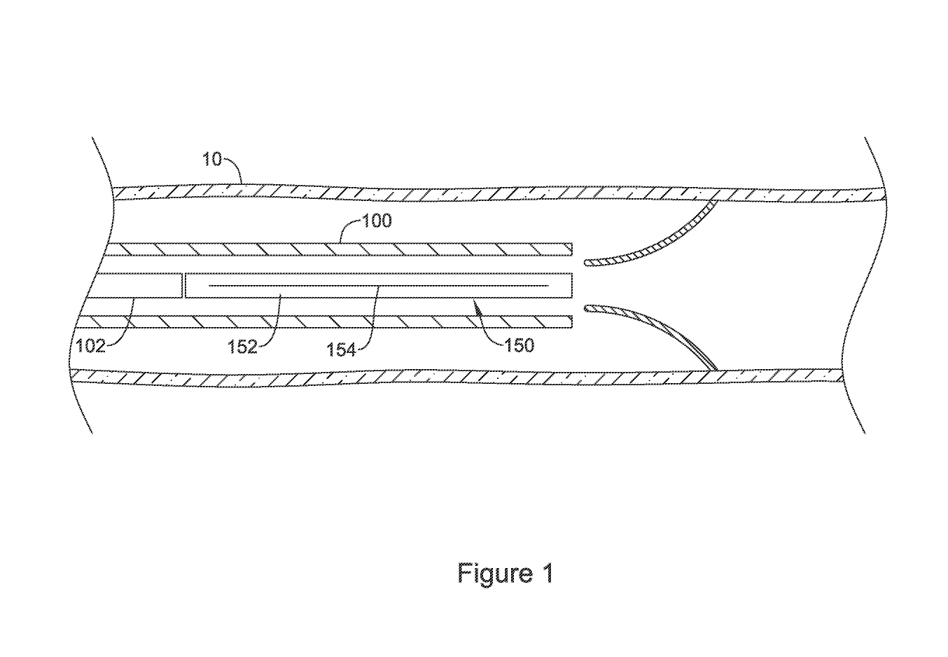

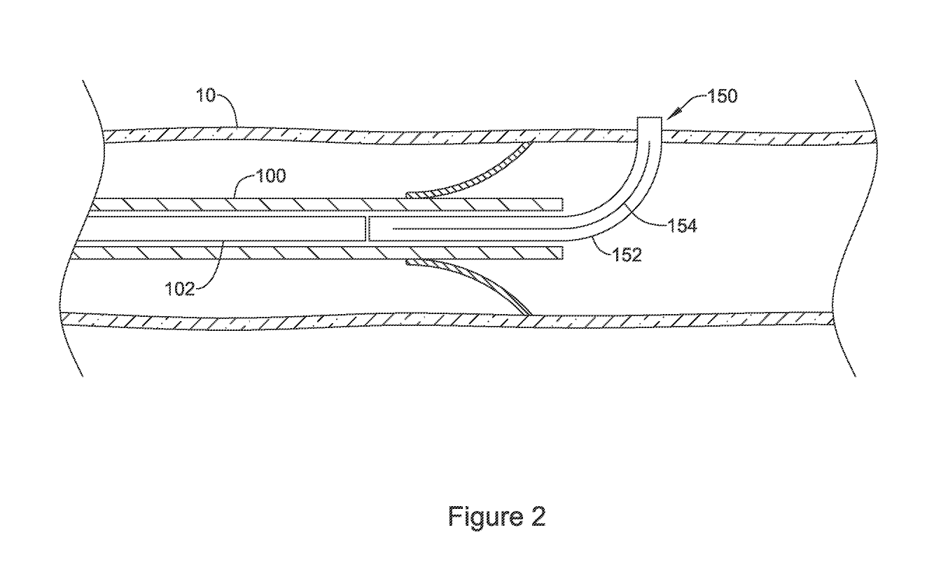

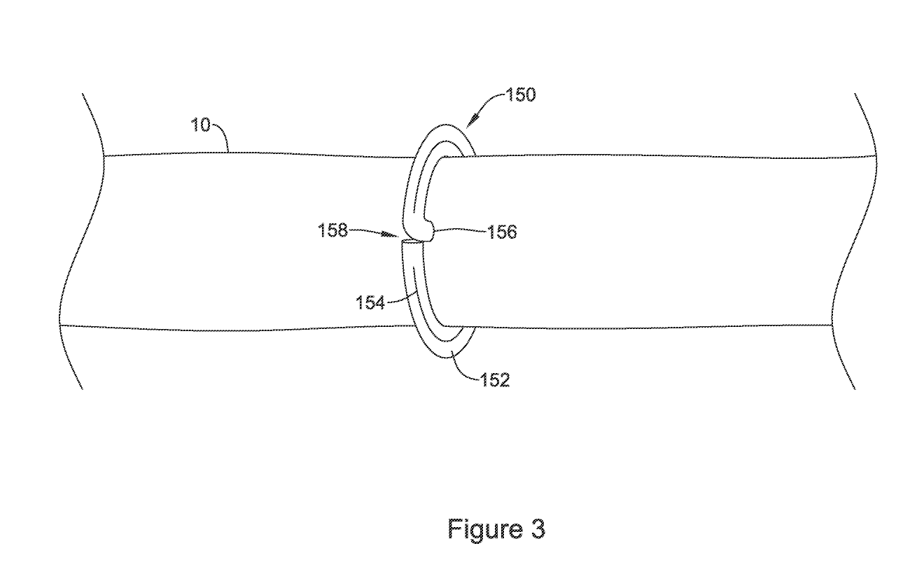

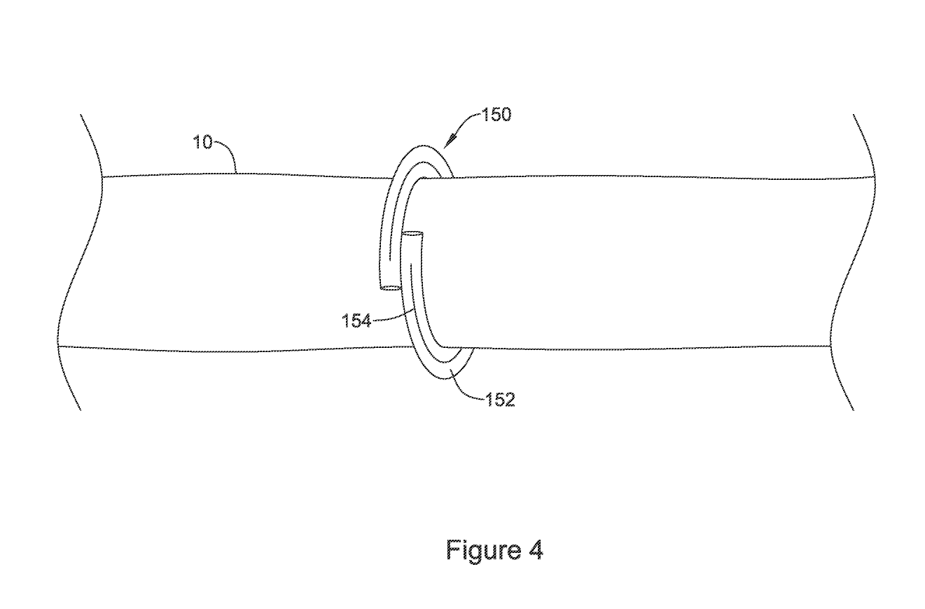

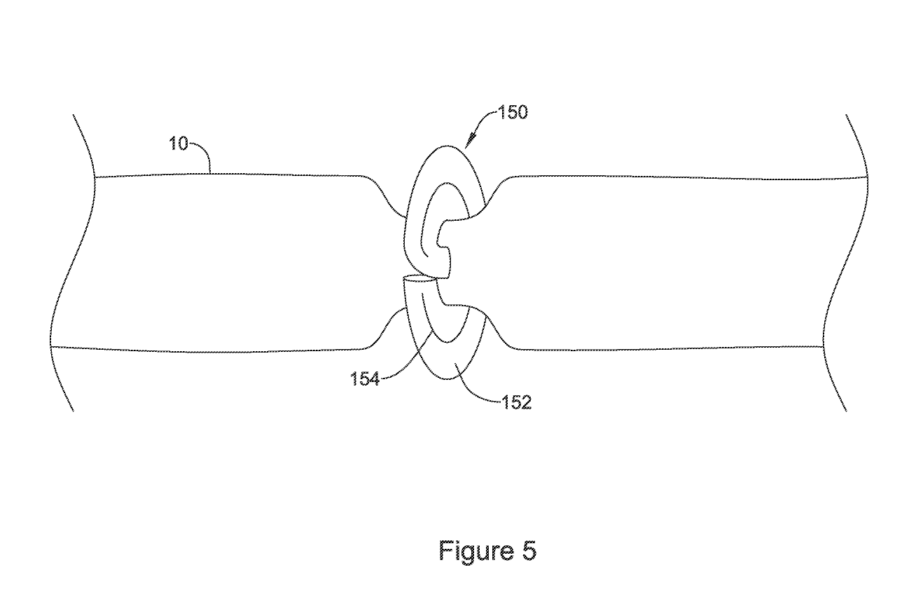

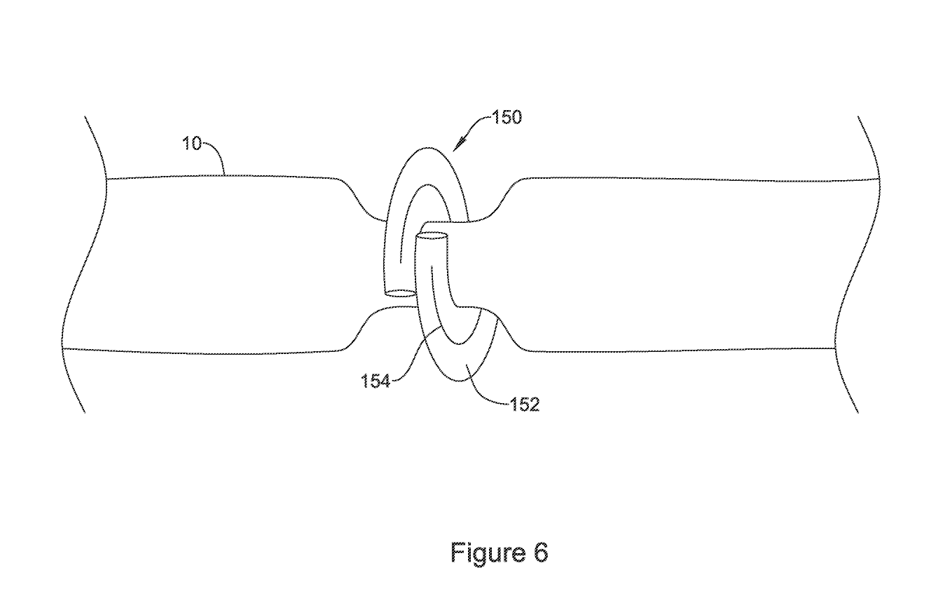

In some embodiments, a percutaneously-implantable extravenous valve may be inserted and/or delivered to a treatment location percutaneously through a body lumen 10, such as a blood vessel (e.g., vein) or other suitable body lumen, through and/or by a delivery sheath 100, as seen in FIGS. 1 and 2 for example. In some embodiments, an extravenous valve may include a constricting member 150 configured to surround an outer wall of a body lumen 10 (i.e., blood vessel, vein, etc.) at the treatment location and actuate between a vessel-occluding and/or lumen-occluding configuration, as seen in FIGS. 5-6 for example, and a non-vessel and/or non-lumen-occluding occluding configuration, as seen in FIGS. 3-4 for example. In some embodiments, the constricting member 150 may include a base material 152 and a shape memory material 154 coupled to the base material 152. In some embodiments, the shape memory material 154 may be at least partially embedded in the base material 152. In some embodiments, the shape memory material 154 may be completely embedded in the base material 152. In some embodiments, the shape memory material 154 may be disposed on an outer surface of the base material 152. In some embodiments, the shape memory material 154 may be self-biased toward a configuration capable of surrounding and/or encircling the outer wall of the body lumen 10 (e.g., blood vessel, vein, etc.).

in some embodiments, the constricting member 150 may be sized and configured to be delivered to the treatment location through the body lumen 10 (e.g., blood vessel, vein, etc.). In some embodiments, the constricting member 150 may be fixedly attachable to an outer surface of the body lumen 10 (e.g., blood vessel, vein, etc.) at a first end. In some embodiments, the constricting member 150 may be releasably attachable to an outer surface of the body lumen 10 (e.g., blood vessel, vein, etc.). In some embodiments, the first end of the constricting member 150 may include an anchoring means 156. In some embodiments, the anchoring means 156 may include a mechanical anchoring element, a surgical glue, adhesive, or other bonding agent, or other suitable anchoring structure. In general, an outer surface of the constricting member 150 may be slippery and/or may include a coating as described below, so as to have a low coefficient of friction against the outer wall of the body lumen 10 (e.g., blood vessel, vein, etc.).

In some embodiments, an in-vivo latching and/or connecting mechanism 158 may be provided to enable percutaneous deployment of the constricting member 150. In some embodiments, the in-vivo latching and/or connecting mechanism 158 may permit a second end of the constricting member 150 to be latched to, connected to, attached to, welded to, bonded to, and/or otherwise coupled to the first end of the constricting member 150. In some embodiments, the in-vivo latching and/or connecting mechanism 158 may permit the second end of the constricting member 150 to be latched to, connected to, attached to, welded to, bonded to, and/or otherwise coupled to the first end of the constricting member 150 in an end-to-end arrangement, as generally seen in FIGS. 3 and 5 for example. In some embodiments, the in-vivo latching and/or connecting mechanism 158 may permit the second end of the constricting member 150 to be latched to, connected to, attached to, welded to, bonded to, and/or otherwise coupled to the first end of the constricting member 150 in an overlapping or side-by-side arrangement, as generally seen in FIGS. 4 and 6 for example. Other configurations, arrangements, and/or combinations thereof are also contemplated. In some embodiments, different arrangements of the constricting member 150 may permit a single size of the extravenous valve to be used with a number of different size (e.g., diameter) body lumens 10 (e.g., blood vessel, vein, etc.).

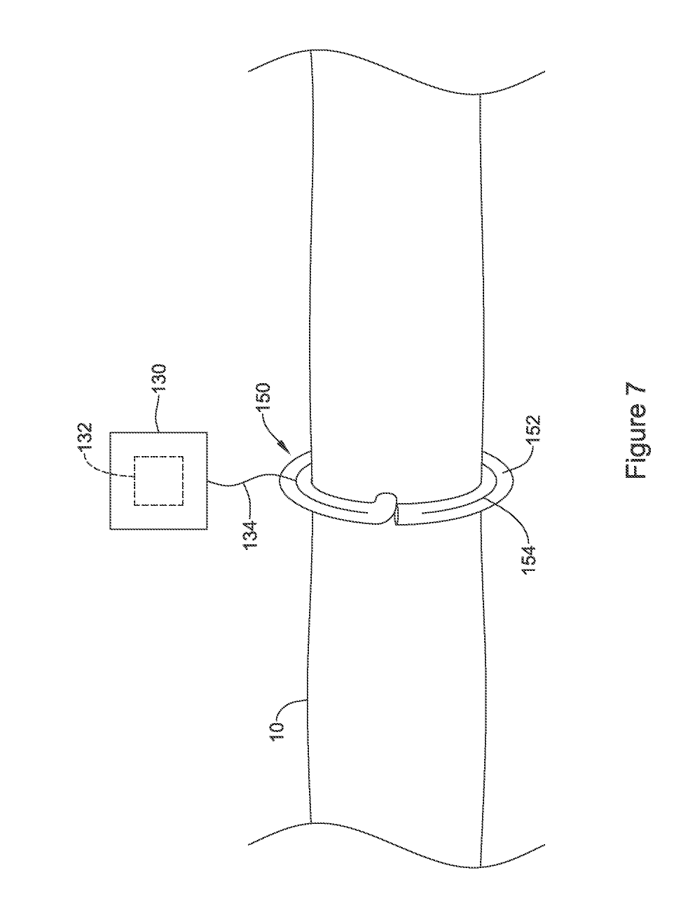

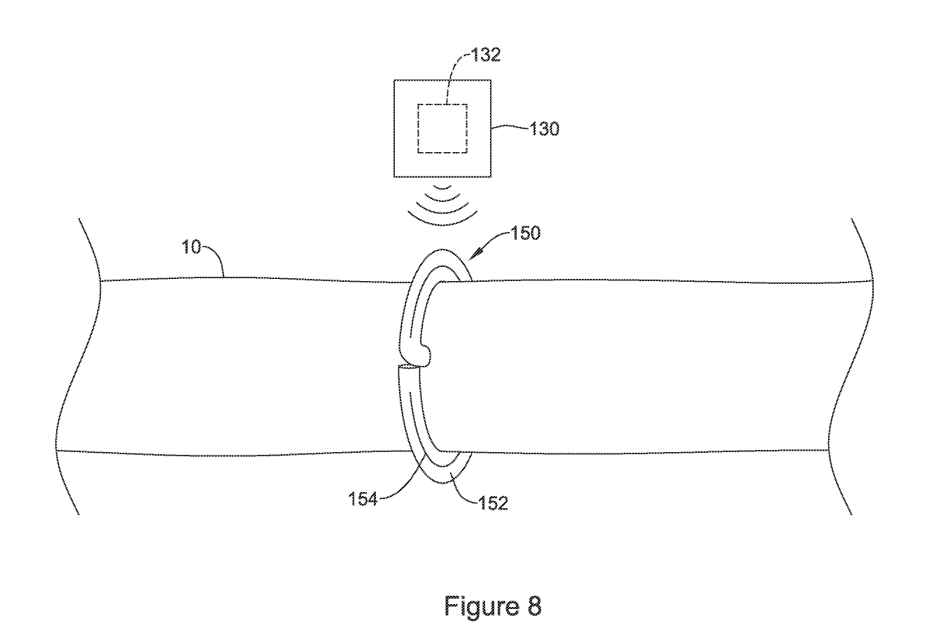

In some embodiments, the extravenous valve may include a power source 130 in communication with the constricting member 150, as seen in FIGS. 7 and 8 for example. In some embodiments, the extravenous valve may include a controller 132 configured to receive bio-feedback from a patient. In some embodiments, bio-feedback includes one or more of the following: blood pressure, heart rate, respiration rate, and oxygenation (of the blood). In some embodiments, the controller 132 may be integral with the power source 130, such as being disposed within a single housing for example. In some embodiments, the controller 132 may be a component or sub-system of the power source 130. In some embodiments, the power source 130 may be a component or sub-system of the controller 132. In some embodiments, the power source 130 and the controller 132 may be separate and distinct components or structures. In some embodiments, the controller 132 may be configured to continuously and/or reversibly actuate the constricting member 150 back and forth between the vessel-occluding and/or lumen-occluding configuration and the non-vessel-occluding and/or non-lumen-occluding configuration in response to the bio-feedback.

In some embodiments, the power source 130 may communicate with the constricting member 150 via one or more wires 134, as seen in FIG. 7 for example. In some embodiments, the power source 130 may communication with the constricting member 150 wirelessly, as seen in FIG. 8 for example. In some embodiments, the power source 130 may be integral with and/or disposed on or within the constricting member 150. In some embodiments, the power source 130 may be disposed remotely from the constricting member 150. In at least some embodiments, the power source 130 may be configured to be carried by the patient. In some embodiments, the power source 130 may include an energy harvesting system. In some embodiments, the power source 130 may include a battery or energy storage unit. In some embodiments, the power source 130 may supply energy to the base material 152, the shape memory material 154, or both.

In some embodiments, energy supplied by the power source 130 may cause heating (e.g., resistive heating, etc.) of the constricting member 150, the base material 152, and/or the shape memory material 154. In some embodiments, heating (e.g., resistive heating, etc.) of the constricting member 150, the base material 152, and/or the shape memory material 154 may result in expansion, contraction, and/or shape change of the constricting member 150, the base material 152, and/or the shape memory material 154. For example, in some embodiments, heating of the shape memory material 154 may result in contraction of the shape memory material 154 and/or expansion of the base material 152. In some embodiments, heating of the shape memory material 154 may result in a shape change of the shape memory material 154.

In some embodiments, energy supplied to and/or heating of the constricting member 150, the base material 152, and/or the shape memory material 154 may cause the constricting member 150 to actuate from a non-vessel-occluding and/or non-lumen occluding configuration to a vessel-occluding and/or lumen-occluding configuration. In some embodiments, energy supplied to and/or heating of the constricting member 150, the base material 152, and/or the shape memory material 154 may cause the constricting member to actuate from a vessel.-occluding and/or lumen occluding configuration to a non-vessel-occluding and/or non-lumen-occluding configuration.

In some embodiments, the controller 132 may be configured to receive bio feedback from a patient, and to actuate the constricting member 150 between the non-vessel-occluding and/or non-lumen occluding configuration and the vessel-occluding and/or lumen-occluding configuration by changing and/or altering the energy supplied to the constricting member 150, the base material 152, and/or the shape memory material 154 in response to the bio-feedback. In some embodiments, the constricting member 150 may be configured to pump a fluid (e.g., blood, etc.) through the body lumen 10 (e.g., blood vessel, vein, etc.) using peristaltic motion when reversibly and continuously actuated back and forth between the non-vessel-occluding and/or non-lumen occluding configuration and the vessel-occluding and/or lumen-occluding configuration.

In use, an extravenous valve may be advanced to a treatment location of a body lumen 10, such as a body lumen 10 of the vascular, urinary, biliary, tracheobronchial, esophageal, or renal tracts. In some embodiments, the extravenous valve may be introduced into and/or advanced percutaneously through a patient's vasculature within a lumen of a delivery sheath 100 in a delivery configuration (as seen in FIG. 1) to a treatment location of a patient's body lumen 10 (e.g., a lumen of a blood vessel having a defective venous valve, for example). In some embodiments, the delivery sheath 100 may include a plunger 102 disposed within the lumen of the delivery sheath 100 proximal of the constricting member 150, which may be disposed within a distal end of the delivery sheath 100. In some embodiments, the plunger 102 may be configured to push and/or advance the constricting member 150 distally out an open distal end of the delivery sheath 100 when the plunger 102 is translated distally.

In some embodiments, the delivery sheath 100 may be advanced to a treatment location of a body lumen 10 (e.g., blood vessel, vein, etc.) in a first direction within the body lumen 10. In some embodiments, the first direction may be a superior-to-inferior direction, a downstream-to-upstream direction, or another suitable direction. In some embodiments, the first direction may be an inferior-to-superior direction, an upstream-to-downstream direction, or another suitable direction. In some embodiments, the first direction may be selected based upon and/or may depend upon one or more factors such as (but not limited to) the treatment location, the access point to the body lumen 10, and/or the size of the body lumen 10 at the access point and/or the treatment location. For the purpose of exemplary discussion herein, the first direction may be illustrated as a downstream-to-upstream direction, and the treatment location may be illustrated as a vein in a lower limb (e.g., leg). However, the skilled practitioner will readily recognize that the discussion herein may be applied and/or adapted for use with a different first direction and/or a different treatment location.

In some embodiments, the delivery sheath 100 may include a puncturing device configured to puncture a hole in a wall of a body lumen 10 (e.g., blood vessel, vein, etc.) at the treatment location. In some embodiments, the puncturing device may be integrally formed with the delivery sheath 100—such as a sharpened distal end, for example. In some embodiments, the puncturing device may be extendible distally from within the delivery sheath 100. In some embodiments, the puncturing device may be disposed within and extendible distally from the lumen of the delivery sheath 100. In some embodiments, the puncturing device may be disposed within and extendible distally from a separate and/or secondary lumen of the delivery sheath 100. In some embodiments, the puncturing device may be extendible distally from the lumen of the delivery sheath 100 in sequence with the constricting member 150 (i.e., the puncturing device may be extended first, and then the constricting member 150 may be advanced after the puncturing device). In some embodiments, the puncturing device may be extendible distally from the lumen of the delivery sheath 100 to puncture the wall of the body lumen 10 (e.g., blood vessel, vein, etc.), withdrawn and removed from the delivery sheath 100 while the delivery sheath 100 is maintained in position within the body lumen 10 (e.g., blood vessel, vein, etc.), and then the constricting member 150 and the plunger 102 may replace the puncturing device within the lumen of the delivery sheath 100 and be advanced distally therein to the treatment location. Other methods and configurations are al so contemplated.

In some embodiments, the distal end of the delivery sheath 100 may be advanced. In the first direction to a position adjacent a treatment location and/or a defective venous valve, the wall of the body lumen 10 (e.g., blood vessel, vein, etc.). In some embodiments, the distal end of the delivery sheath 100 may be positioned upstream of the treatment location and/or a defective venous valve. In some embodiments, the distal end of the delivery sheath 100 may be positioned downstream of the treatment location and/or a defective venous valve.

After advancing the distal end of the delivery sheath 100 to a position adjacent a treatment location and/or a defective venous valve, the wall of the body lumen 10 (e.g., blood vessel, vein, etc.) may be punctured adjacent the treatment location and/or the defective venous valve. In some embodiments, the wall of the body lumen 10 (e.g., blood vessel, vein, etc.) may be punctured upstream of the treatment location and/or the defective venous valve. In some embodiments, the wall of the body lumen 10 (e.g., blood vessel, vein, etc.) may be punctured downstream of the treatment location and/or the defective venous valve. In some embodiments, the distal end of the delivery sheath 100 may be positioned adjacent the puncture in wall of the body lumen 10 (e.g., blood vessel, vein, etc.). Next, the constricting member 150 may be delivered out the open distal end of the delivery sheath 100 through the puncture to an exterior of the body lumen 10 (e.g., blood vessel, vein, etc.) by advancing the plunger 102 distally to push the constricting member 150 out the open distal end of the delivery sheath 100.

In some embodiments, the constricting member 150 may be configured to surround the body lumen 10 (e.g., blood vessel, vein, etc.) adjacent an exterior surface of the body lumen 10 (e.g., blood vessel, vein, etc.) and/or self-biased toward a configuration capable of encircling or surrounding the body lumen 10 (e.g., blood vessel, vein, etc.), as seen in FIGS. 3 and 4 for example. After passing through the puncture and/or the wall of the body lumen 10 (e.g., blood vessel, vein, etc.), the constricting member 150 may encircle and/or surround the body lumen 10 (e.g., blood vessel, vein, etc.) as the constricting member 150 is advanced out the open distal end of the delivery sheath 100. In some embodiments, the constricting member 150 may be positioned upstream of the treatment location and/or the defective venous valve. In some embodiments, the constricting member 150 may be positioned downstream of the treatment location and/or the defective venous valve. In some embodiments, the constricting member 150 may be positioned about, around, and/or directly over the treatment location and/or the defective venous valve.

In some embodiments, a first end of the constricting member 150 may include an anchoring means 156 that may be fixedly attachable to an exterior surface of the wall of the body lumen 10 (e.g., blood vessel, vein, etc.), as seen in FIG. 3 for example. In some embodiments, the constricting member 150 may include an in-vivo latching and/or connecting mechanism 158, as also seen in FIG. 3 for example, which may form the constricting member 150 into a generally annular shape. In some embodiments, the constricting member 150 may form a helical or spiral shape around the exterior surface of the body lumen 10 (e.g., blood vessel, vein, etc.), as seen in FIG. 4 for example. In some embodiments, opposing first and second ends of the constricting member 150 may overlap circumferentially, as also seen in. FIG. 4 for example. While not explicitly illustrated, a helically-oriented, circumferentially overlapping constricting member 150, as seen in FIG. 4, may also include an in-vivo latching and/or connecting mechanism 158 as described above. Following delivery and/or deployment of the constricting member 150, the puncture in the wall of the body lumen 10 (e.g., blood vessel, vein, etc.) may be closed and/or sealed. A variety of different methods for closing a vascular puncture are known and need not be described in detail herein. Any suitable method for closing the puncture may be utilized with the current disclosure, although in some embodiments, preference may be given to a percutaneously-based method used in conjunction with the extravenous valve of the current disclosure.

A method of treating a defective venous valve in accordance with the current disclosure may include introducing a delivery sheath 100 into a body lumen 10 and/or a lumen of a blood vessel having a defective venous valve, the delivery sheath 100 having a constricting member 150 of an extravenous valve disposed within a distal end thereof. In some embodiments, the method may include advancing the distal end of the delivery sheath 100 to a position adjacent the defective venous valve. In some embodiments, the method may include advancing the distal end of the delivery sheath 100 in a first direction from downstream-to-upstream within the body lumen 10 and/or the lumen of the blood vessel having the defective venous valve to the position adjacent the defective venous valve.

In some embodiments, the method may include puncturing a wall of the body lumen 10 and/or the blood vessel adjacent the defective venous valve. In some embodiments, the method may include puncturing the wall of the body lumen 10 and/or the blood vessel upstream of the defective venous valve. In some embodiments, the method may include positioning the distal end of the delivery sheath 100 adjacent the puncture in the wall of the body lumen 10 and/or the blood vessel. In some embodiments, the method may include delivering the constricting member 150 out the distal end of the delivery sheath 100 through the puncture, wherein the constricting member 150 is configured to surround the body lumen 10 and/or the blood vessel adjacent an exterior surface of the body lumen 10 and/or the blood vessel. In some embodiments, the method may include continuously actuating the constricting member 150 between a vessel-occluding configuration and a non-vessel-occluding configuration.

In some embodiments, a constricting member 150 in accordance with the method described herein may include a base material 152 and a shape memory material 154 coupled to the base material 152. On some embodiments, the extravenous valve in accordance with the method described herein may include a power source 130 and a controller 132 in communication with the constricting member 150. In some embodiments, the method may include receiving bio-feedback with the controller 132. In some embodiments, the method may include continuously actuating the constricting member 150 between the vessel-occluding configuration and the non-vessel-occluding configuration in response to the bio-feedback. In some embodiments, the method may include anchoring a first end of the constricting member 150 to the body lumen 10 and/or the blood vessel. In some embodiments, the method may include closing and/or sealing the puncture in the wall of the body lumen 10 and/or the blood vessel.

In some embodiments, the base material 152 and/or the delivery sheath 100 may be a biocompatible polymer. Some examples of suitable polymers may include polytetrafluoroethylene (PTFE), ethylene tetrafluoroethylene (ETFE), fluorinated ethylene propylene (FEP), polyoxyrnethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRESTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (P11), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidate chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In some embodiments, the shape memory material 154 may include a suitable metallic alloy, such as but not limited to nitinol, nickel-titanium alloy such as linear-elastic and/or super-elastic nitinol, other nickel-based alloys, a shape memory polymer, an electro-active polymer, or other suitable material having an ability to change shape, configuration, and/or state.

In same embodiments, an exterior surface of the extravenous valve, the constricting member 150, and/or the delivery sheath 100 may include a coating, for example a lubricious, a hydrophilic, a protective, or other type of coating may be applied over portions or all of the extravenous valve, the constricting member 150, and/or the delivery sheath 100. Alternatively, the extravenous valve, the constricting member 150, and/or the delivery sheath 100 may comprise a lubricious, hydrophilic, protective, or other type of coating. Hydrophobic coatings such as fluoropolymers provide a dry lubricity which improves device handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. In some embodiments, the extravenous valve, the constricting member 150, the delivery sheath 100, and/or a coating disposed thereon may include a therapeutic agent disposed thereon and/or embedded therein.

In at least some embodiments, portions or all of the extravenous valve, the constricting member 150, and/or the delivery sheath 100 may also be doped with, made of, or otherwise include a radiopaque material. Radiopaque materials are understood to be materials capable of producing a relatively bright image on a fluoroscopy screen or another imaging technique during a medical procedure. This relatively bright image aids the user in determining the location of the extravenous valve, the constricting member 150, and/or the delivery sheath 100. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the extravenous valve, the constricting member 150, and/or the delivery sheath 100 to achieve the same result.

In some embodiments, a degree of Magnetic Resonance imaging (MRI) compatibility is imparted into the extravenous valve, the constricting member 150, and/or the delivery sheath 100. For example, the extravenous valve, the constricting member 150, and/or the delivery sheath 100, or portions thereof, may be made of a material that does not substantially distort the image and create substantial artifacts (i.e., gaps in the image). Certain ferromagnetic materials, for example, may not be suitable because they may create artifacts in an MRI image. The extravenous valve, the constricting member 150, and/or the delivery sheath 100, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

An exemplary extravenous valve and/or constricting member 150 may be configured to be positioned at a treatment location for a variety of medical applications. For example, the extravenous valve and/or the constricting member 150 may be used to treat a stenosis in a blood vessel, used to actuate a fluid pathway in the vascular, urinary, biliary, tracheobronchial, esophageal, or renal tracts, or other body lumens, in some instances. Although illustrated herein as being introduced or delivered percutaneously, the extravenous valve and/or the constricting member 150 may also be introduced or delivered endoscopically, subcutaneously, and/or surgically to be positioned within, on, and/or around an organ, tissue, or body lumen, such as a heart, artery, vein, urethra, esophagus, trachea, bronchus, bile duct, or the like.

Those skilled in the art will recognize that aspects of the present disclosure may be manifested in a variety of forms other than the specific embodiments described and contemplated herein. Accordingly, departure in form and detail may be made without departing from the scope and spirit of the present disclosure as described in the appended claims. This may include, to the extent that it is appropriate, the use of any of the features of one example embodiment or aspect being used in other embodiments or aspects.

What is claimed is:

1. An extravenous valve, comprising:
a constricting member configured to surround a blood vessel and actuate between a vessel-occluding configuration and a non-vessel-occluding configuration, the constricting member including a base material and a shape memory material coupled to the base material;
a power source in communication with the constricting member; and
a controller configured to receive bio-feedback from a patient;
wherein the controller reversibly actuates the constricting member between the vessel-occluding configuration and the non-vessel-occluding configuration in response to the bio-feedback.

2. The extravenous valve of claim 1, wherein the controller continuously actuates the constricting member back and forth between the vessel-occluding configuration and the non-vessel-occluding configuration.

3. The extravenous valve of claim 1, wherein the base material is a polymer.

4. The extravenous valve of claim 1, wherein the shape memory material is at least partially embedded within the base material.

5. The extravenous valve of claim 1, wherein the power source communicates with the constricting member via one or more wires.

6. The extravenous valve of claim 1, wherein the power source communicates with the constricting member wirelessly.

7. The extravenous valve of claim 1, wherein the constricting member is fixedly attachable to the blood vessel at a first end.

8. The extravenous valve of claim 7, wherein the first end includes an anchoring means.

9. The extravenous valve of claim 1, wherein the shape memory material is self-biased toward a configuration capable of encircling the blood vessel.

10. The extravenous valve of claim 1, wherein the power source is carried by the patient.

11. The extravenous valve of claim 1, wherein the constricting member is configured to pump blood through the blood vessel.

12. A percutaneously-implantable extravenous valve, comprising:
a constricting member configured to surround an outer wall of a body lumen of a patient at a treatment location and actuate between a lumen-occluding configuration and a non-lumen-occluding configuration, the constricting member including a base material and a shape memory material coupled to the base material;
a power source in communication with the constricting member; and
a controller configured to continuously actuate the constricting member between the lumen-occluding configuration and the non-lumen-occluding configuration;
wherein the constricting member is sized and configured to be delivered to the treatment location through the body lumen.

13. The extravenous valve of claim 12, wherein the controller is configured to receive bio-feedback from the patient.

14. The extravenous valve of claim 13, wherein the controller is configured to continuously actuate the constricting member between the lumen-occluding configuration and the non-lumen-occluding configuration in response to the bio-feedback.

15. The extravenous valve of claim 13, wherein the bio-feedback includes one or more of blood pressure, heart rate, respiration rate, and oxygenation.

16. A method of treating a defective venous valve, comprising:
introducing a delivery sheath into a lumen of a blood vessel having a defective venous valve, the delivery sheath having a constricting member of an extravenous valve disposed within a distal end thereof;
advancing the distal end of the delivery sheath to a position adjacent the defective venous valve;
puncturing a wall of the blood vessel adjacent the defective venous valve;
positioning the distal end of the delivery sheath adjacent the puncture;
delivering the constricting member out the distal end of the delivery sheath through the puncture, wherein the constricting member is configured to surround the blood vessel adjacent an exterior surface of the blood vessel; and
continuously actuating the constricting member between a vessel-occluding configuration and a non-vessel-occluding configuration.

17. The method of claim 16, wherein the constricting member includes a base material and a shape memory material coupled to the base material.

18. The method of claim 17, wherein the extravenous valve includes a power source and a controller in communication with the constricting member.

19. The method of claim 18, further including:
receiving bio-feedback with the controller; and
continuously actuating the constricting member between the vessel-occluding configuration and the non-vessel-occluding configuration in response to the bio-feedback.

20. The method of claim 17, further including anchoring a first end of the constricting member to the blood vessel.
